(19) United States
(12) Patent Application Publication
(10) Pub. No.: US 2024/0016981 A1
(43) Pub. Date: Jan. 18, 2024

(54) REGENERATIVE TISSUE MANUFACTURING PROCESS

(71) Applicant: Vascudyne, Inc., St. Paul, MN (US)

(72) Inventor: Richard F. Murphy, White Bear Township, MN (US)

(21) Appl. No.: 18/256,867

(22) PCT Filed: Dec. 9, 2021
(86) PCT No.: PCT/US2021/062709

§ 371 Date: Jun. 9, 2023

Related U.S. Application Data
(60) Provisional application No. 63/123,929, filed on Dec. 10, 2020.

(51) Int. Cl. A61L 27/36; A61L 27/50; A61L 27/22

(52) U.S. Cl. CPC A61L 27/3633; A61L 27/3683; A61L 27/507; A61L 27/225

(57) ABSTRACT

The present invention is an improved implant made from regenerative tissue or natural tissue, methods of making the implant, and methods of using the implant.

### FIELD OF THE INVENTION

The invention relates to a method for manufacturing regenerative tissue, and an implant comprising this tissue.

### BACKGROUND OF THE INVENTION

A relatively new field of medicine—since the early 1990s—is the field of Regenerative Medicine. Regenerative Medicine is the process of creating functional tissues to repair, replace, or restore tissue or organ structure and function lost due to age, disease, damage, or congenital defects. This field of medicine uses new methods including (stem) cell therapy, development of medical devices, and tissue engineering.

The use of prepared heterogenous graft material for human surgical implantation is well known. More specifically, the use of treated animal tissue as human tissue grafts, replacement valves, and similar implantation surgical procedures is well known. However, problems of immunogenicity, thrombogenicity, calcification, material strength, and size have not been adequately addressed in the prior art.

Since the 1930's, medical researchers have attempted to develop suitable natural and synthetic alternatives for obtaining small diameter grafts useful in vascular surgery. Historically, attempts to fabricate such tubular grafts from man-made materials have been somewhat unsuccessful. Homologous tissues, however, are not always readily available, and are not always readily available in the size the surgeon needs. Furthermore, some of these tissues may be immunogenic and therefore may require processing or certain treatments to reduce their immunogenicity.

One of the problems encountered, for both natural and synthetic grafts, is producing a small diameter graft, e.g., less than about 6 mm internal diameter. For small diameter grafts made of heterologous or synthetic material, as the diameter of the graft decreases, the opportunity for blockage increases due to the inherent thrombogenicity of such materials. Moreover, for all grafts of a small diameter, as the size decreases, the sutureability and flexibility of the graft may decrease, both characteristics that are highly desirable in a suitable graft material. For these and other reasons, the conventional grafts are typically 6 to 10 mm (an intermediate size graft) or greater than 10 mm (a large graft).

Notwithstanding the usefulness of the above-described methods, a need still exists for increasing patency; making an implant less thrombotic in structure and/or function; minimizing calcification; and increasing the useful life of the implant.

### SUMMARY OF THE INVENTION

One embodiment of the invention is the preparation and use of RT to make implants and their use as implants.

An advantage of both embodiments of the invention is the starting material itself.

The biological materials according to the present invention, are processed to modify (e.g., reduce or eliminate) calcification characteristics, resorbability, size and shape, thinness, collagen content, and other characteristics and properties that will become clear from the description of the invention. The methods, uses, and products of the present invention are intended for implant in a mammal, preferably a human. All of the biological materials, processed according to the present invention, are appropriate for use in an in vivo environment, and include one or more of the following desirable properties for graft material suitable for implantation: a) size compatibility with surrounding vessels to which it will be anastomosed; b) sutureability, kink resistance, softness, radial and longitudinal compliance, and flexibility (a softer hand); c) non-thrombogenicity or low levels of thrombogenicity; d) durability; e) ease of sterilization; f) readily available, and available in diameters and lengths appropriate for surgical procedures; g) shelf life appropriate for market conditions (typically greater than three years); h) resistant to infection; i) sufficient strength to resist aneurysm formation; j) non-immunogenic; k) resistant to degradation; and l) resistant to formation of neointimal hyperplasia.

Although specific advantages have been enumerated above, various embodiments may include some, none, or all of the enumerated advantages.

Other technical advantages may become readily apparent to one of ordinary skill in the art after review of the following figures and description.

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

With the following enabling description of the drawings, the apparatus should become evident to a person of ordinary skill in the art.

### BRIEF DESCRIPTION OF THE FIGURES

FIG. 1 shows generally the cell culture phase during which the cells are grown and expanded, and various ingredient suspensions are formed.

FIG. 2 shows the continuation of the cell culture stage and the expansion of cells through a number of passages.

FIG. 3 shows generally the casting phase and the media change phase during which various suspensions and solutions are combined, the gel begins to form and is transferred to a form, mandrel, or incubator.

FIG. 4 illustrates tissue formation around a form and the processing steps that occur as the cellularized tissue matrix forms.

FIG. 5 illustrates generally the flow chart of the regenerative tissue processing steps.

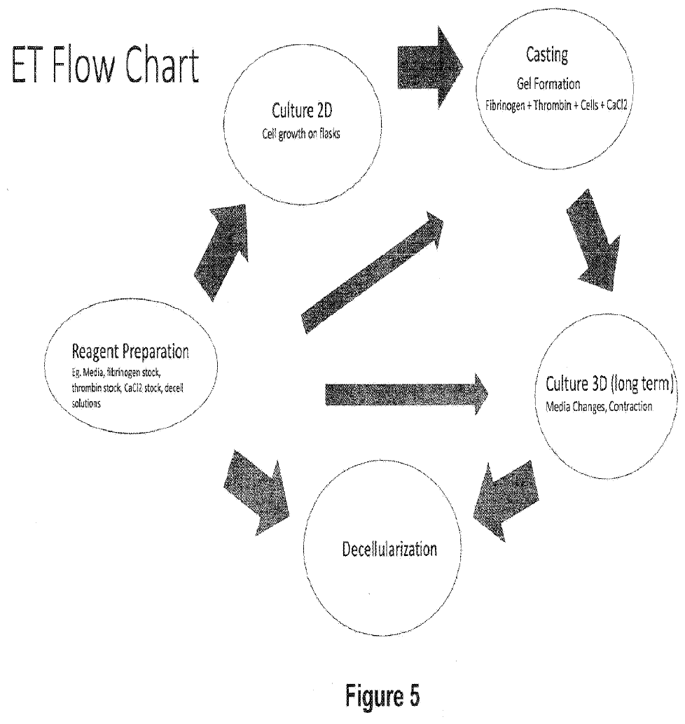

FIG. 6 shows regenerative tissue fiber alignment, tensile strength, and suture retention in comparison to fixed pericardium.

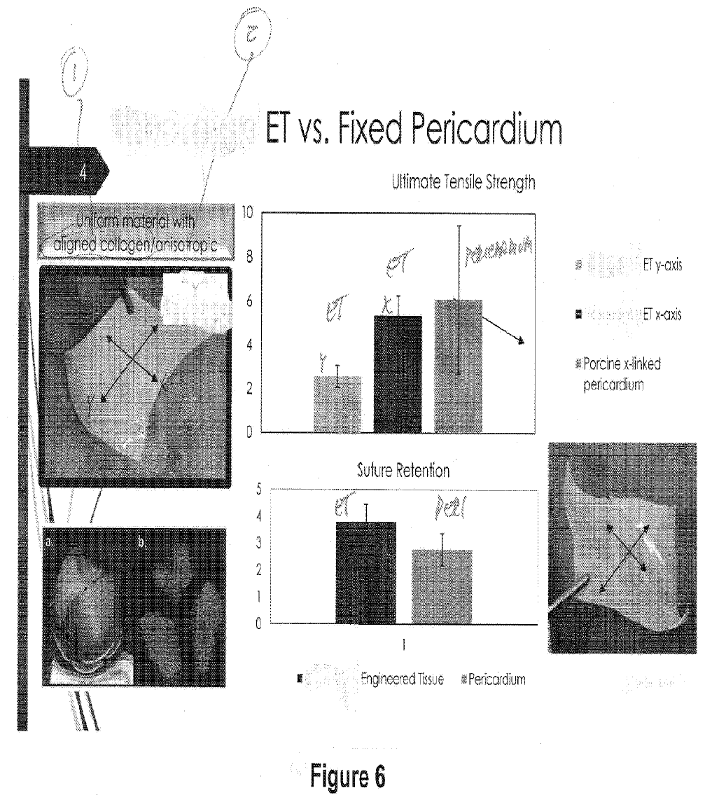

### DETAILED DESCRIPTION OF THE INVENTION

The present invention is a graft or prosthesis formed from regenerative tissue. The invention includes methods of making the tissue and methods of making the graft or prosthesis.

The present invention is also a graft, tube, conduit, or sheet, including but not limited to a bypass graft, a bypass graft for peripheral application; a coronary artery bypass graft; a sheet; and a valve. The graft may be formed by combining ECM-producing cells in the presence of fibrinogen and thrombin under conditions that permit the formation of regenerative tissue. The tissue is then cultured until it matures, e.g., is substantial enough to be used for its intended purpose.

The present invention is also a method of producing or forming the graft, tube, conduit, or sheet.

The present invention is also the use of the graft, tube, conduit, or sheet as an implantable prosthesis, portion of an implantable prothesis (i.e. dura patch, valve leaflets, vascular conduit, etc.).

The present invention is also the use of regenerative tissue, as defined and produced herein, to treat a human or animal disease or condition, including but not limited to cardiovascular diseases and disorders; vascular disease and disorders; and any disease and disorder that may benefit from catheter-based or supplied implantable prosthetics.

A preferred embodiment of the invention is any tubular structure formed from RT, including but not limited to a tubular graft. As described in more detail below, the preferred tubular graft is formed on a 3-D platform, such as a cylinder.

An embodiment of the invention is the use of RT in a bypass graft. In preferred embodiments, the bypass graft is a peripheral bypass graft. Other embodiments of the invention include peripheral bypass grafts above the knee, behind the knee, below the knee, and combinations thereof.

An embodiment of the invention includes an arterio-venous graft, including but not limited to an AV graft used in dialysis.

In accordance with the present invention, any prosthesis may be formed using regenerative tissue (RT) or engineered tissue. Engineered tissue, as used herein, refers to tissue formed or processed as disclosed in the following: 2007/061800; WO 2007/092902; 2016/0203262; WO/2004/018008; WO 2004/101012; U.S. Pat. Nos. 10,105,208; 8,192,981; 8,399,243; 8,617,237; 8,636,793; 9,034,333; 9,126,199; U.S. Ser. No. 16/045,220, filed Jul. 25, 2018 (Tranquillo continuation of U.S. Pat. No. 10,105,208, now allowed); U.S. Ser. No. 10/523,618; U.S. Ser. No. 10/556,959; U.S. Ser. No. 13/771,676; 2015/0012083; 2009/0319003; 2011/0020271; 2012/0230950; 2013/0013083; 2014/0330377; 2014/035805; 2017/0135805; 2017/0296323; 2017/0306292; U.S. Pat. Nos. 8,198,245; 9,127,242; 9,556,414; 9,657,265; and 9,650,603; all of which are hereby incorporated in the entirety be reference.

In one embodiment of the invention, the bioengineered tissue may be made according to U.S. Pat. Nos. 10,111,740; 10,105,208; and U.S. Ser. No. 16/045,220, filed Jul. 25, 2018 (continuation of U.S. Pat. No. 10,105,208, now allowed), all Tranquillo, et al., each incorporated in its entirety be reference. Any process or method for producing engineered tissue is included within the scope of the present invention.

The present invention also is the use of the tissue as an implantable prosthesis. Suitable uses include but are not limited to coronary artery bypass graft (CABG), a valve leaflet, a peripheral graft, a peripheral bypass graft; an AV graft (arteriovenous); a sling, bulking agents, prolapsed bladder repair, stented or stentless pericardial valve replacement, stented or stentless pulmonic valve replacement, transcatheter valve prosthesis, aortic bioprosthesis/valve replacement or repair, annuplasty rings, bariatric surgery, dural patching, enucleation wraps, gastric banding, herniation repair, lung surgery e.g. lung volume reduction, peripheral arterial or venous valve replacement, pericardial patching, rotator cuff repair, uretheral slings, valve repair, vascular patching, valve conduit insertion, or arterial conduit insertion and/or repair.

The regenerative tissue (RT) of the present invention, may be characterized by modifying (e.g., reduce or eliminate) inflammation, calcification characteristics, resorbability, size and shape, thinness (e.g. dilatation or aneurysm formation), collagen content, and other characteristics and properties that will become clear from the description of the invention.

The methods, uses, and products of the present invention are intended for implant in a mammal, preferably a human.

All of the RT, processed according to the present invention, are appropriate for use in an in vivo environment, and include one or more of the following desirable properties: a) size compatibility with surrounding vessels to which it will be anastomosed; b) sutureability, kink resistance, softness, radial and longitudinal compliance, and flexibility (a softer hand); c) non-thrombogenicity or low levels of thrombogenicity; d) durability; e) readily available, and available in diameters and lengths appropriate for surgical procedures; f) shelf life appropriate for market conditions (typically greater than three years); g) resistant to infection; h) sufficient strength to resist aneurysm formation; i) non-immunogenic; j) resistant to degradation; and k) resistant to formation of neointimal hyperplasia.

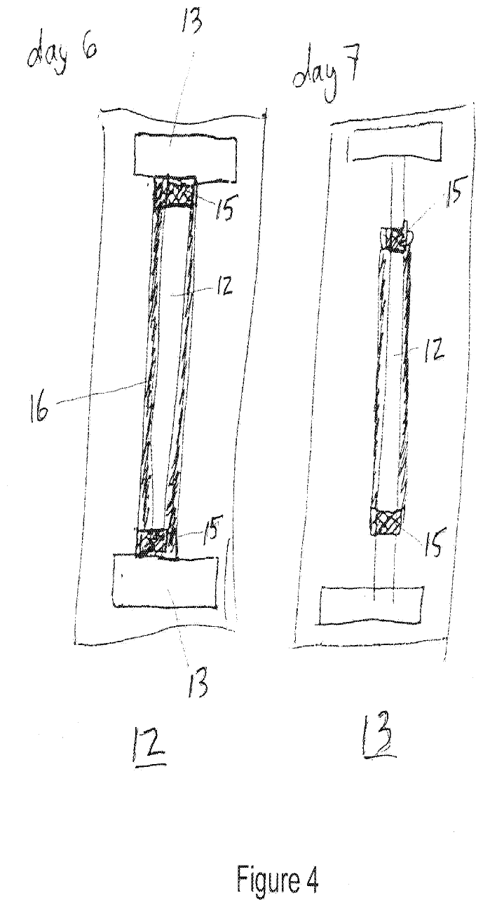

The RT of the present invention is distinct from certain other kinds of regenerative or engineered tissue in the use of crosslinked fibrinogen that is later degraded during the culturing process. Also, the RT of the present invention can be contracted or allowed to contract, for example, in the longitudinal direction and/or in the radial direction, among others. In accordance with some embodiments of the invention, the fibers in the tissue may align or become aligned, believed to be partially due to fibrin having no or little resistance to contraction that occurs naturally as part of the collagen/ECM formation process. The inventors also believe that radial and/or longitudinal contraction occurs in part naturally as an inherent function of tissue forming as described herein. In accordance with a preferred embodiment of the invention, contraction, whether natural or not, may be controlled or retarded, an example of which is shown in FIG. 4, steps 12 and 13. In another embodiment of the invention, the contraction may be scalable or intentionally controlled to enhance, promote, or achieve one or more tissue characteristics, e.g., fiber alignment, or tensile strength, or suturability. Furthermore, the RT of the present invention does not include any synthetic materials, as is typical in other processes that use PLA or PGA or the like.

A product and/or method of the present invention typically includes combining fibrinogen or fibrinogen-like material, thrombin, and matrix-producing cells to produce a fibrin gel with a homogeneous cell suspension. In preferred embodiments of the invention, the cell infused fibrin gel undergoes casting, used herein to refer to encapsulating cells in a fibrin gel, and culturing to form the collagenous tissue or grafts. In other preferred embodiments, the tissue or graft may be allowed to contract (e.g. longitudinally or radially), preferably in a controlled manner. In accordance with the present invention, the process permits customized or optimized fiber alignment during the contraction phase. Customized or optimized alignment includes, but is not limited to radial alignment, longitudinal alignment, both radial and longitudinal alignment, and a pre-determined ratio of radial and longitudinal alignment.

One or more methods of the present invention may also include molding or forming the cell-seeded fibrin gel into a pre-determined shape; manipulating, mechanically and/or manually, the tissue in the presence of culture medium to produce RT; manipulating the tissue during the culturing phase of the tissue; manipulating the tissue during the maturation phase of the tissue; manipulating the tissue during the culturing/maturation phase of the tissue production process; manually moving the graft to evenly distribute the stress relief from the contracting ends; decellularizing the RT; and automated or semi-automated versions of any of the method steps.

By making the tissue manipulation device from a non-adherent material or a material that binds cells only weakly, such as stainless steel or ePTFE, the tissue may be allowed to contract, preferably in a controlled or manual manner, without damaging the tissue.

Each of these ingredients and steps will now be described in more detail.

Cell Source

Cells or a cell line of the present invention is any cell or line that produces an extracellular matrix (ECM). Matrix-producing cells include, but are not limited to fibroblasts, embryonic stem cells, post-natal stem cells, adult stem cells, mesenchymal cells, interstitial cells, endothelial cells, smooth or skeletal muscle cells, myocytes (muscle stem cells), chondrocytes, adipocytes, fibromyoblasts, and ectodermal cells, including ductile and skill cells, hepatocytes, Islet cells, cells present in the intestine and other parenchymal cells, and osteoblasts and other cells forming bone or cartilage.

One skilled in the art will recognize that cells may obtained commercially, ready to use (e.g., pure or substantially pure; and ready to be suspended in media). Typically, the frozen cells are thawed, plated into culture flasks, grown, and harvested. The harvested cells are then ready to be suspended in media, preferably without FBS, and combined with fibrinogen and thrombin solutions.

One skilled in the art will also recognize that cells may need to be isolated or purified. The methods of obtaining a cell population in this and the previous paragraph are well known.

Fibrinogen Source

Any source of fibrinogen may be used. Fibrinogen is typically used in the present invention in a buffer. In preferred embodiments, the fibrinogen is mixed into Hepes buffer.

Thrombin Source

Any source of thrombin may be used. Thrombin is typically used in the present invention in a buffer and conventional cell culture medium. In preferred embodiments, the thrombin is mixed into DMEM with Hepes.

In the most preferred embodiments, the thrombin solution also includes a clot formation factor or co-factor, such as a source of calcium. The preferred source of calcium is CaCl₂.

Media

One or more matrix-producing cells may be cultured in any nutrient, growth, or maintenance medium. Any cell culture medium may be used. A preferred medium is Eagle's minimal essential medium; a most preferred medium is DMEM (Dulbecco's modified Eagle's medium).

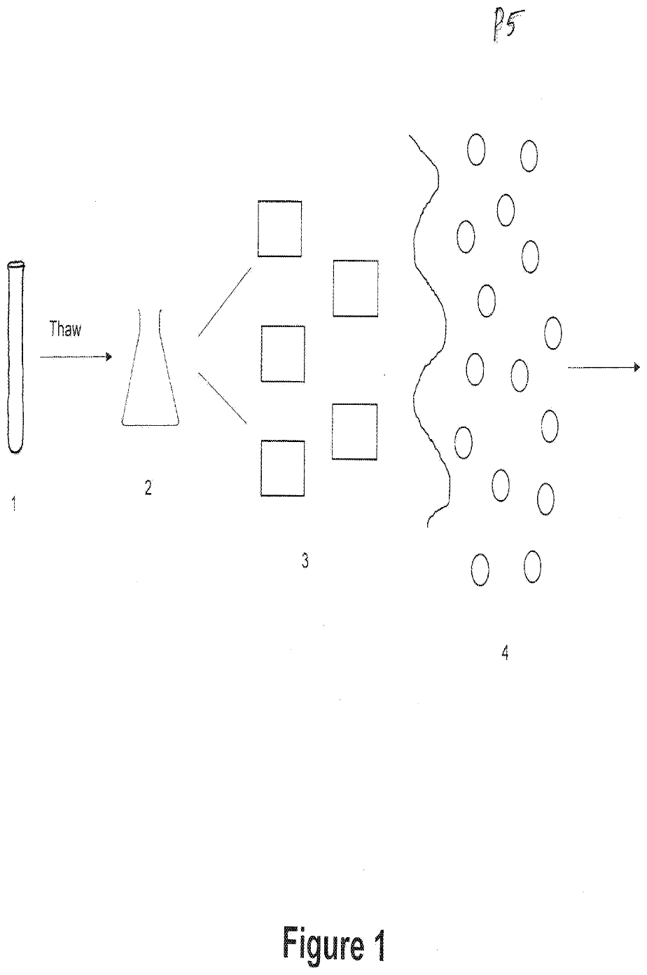

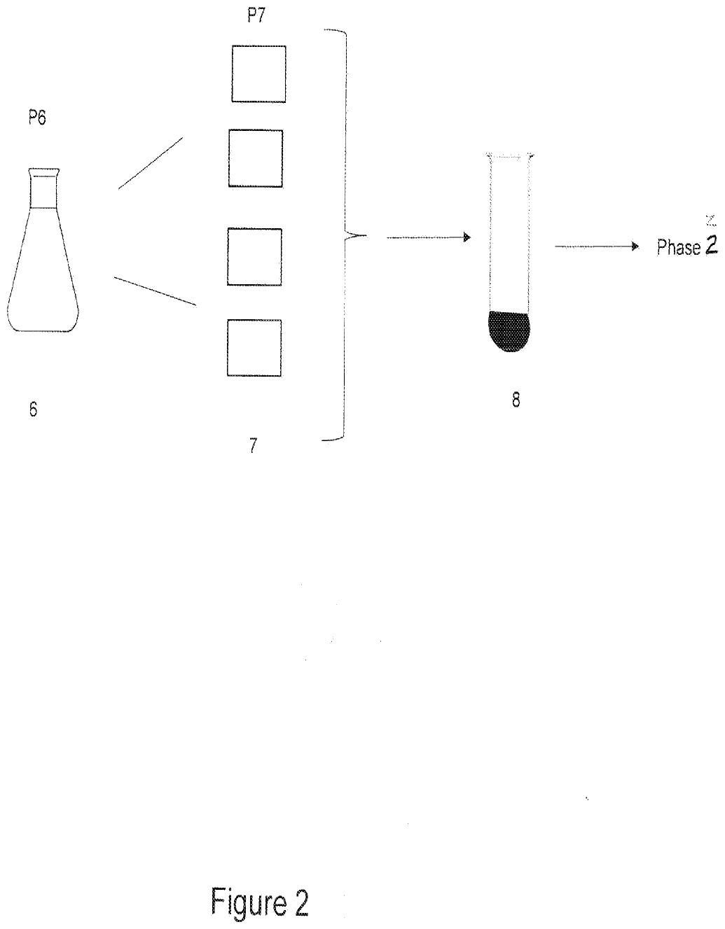

During growth in culture, sometimes referred to as the 2D stage (also, FIGS. 1 and 2), the cells may be cultured with agents that promote cellular proliferation and growth. Such agents include a number of growth factors that can be selected based upon the tissue to be grown and the cell types present (e.g., keratinocyte growth factor (KGF); vascular endothelial cell growth factor (VEGF); platelet derived growth factor (PDGF); fibroblast growth factor (FGF); a transforming growth factor (TGF) alpha, beta, and the like; insulin; growth hormone; somatomedins; colony stimulating factors; erythropoietin; epidermal growth factor; hepatic erythropoietic factor (hepatopoietin); and liver-cell growth factor to name a few, others are known in the art). Serum, such as fetal bovine serum (FBS) or the like, can also provide some of these growth factors. In addition, agents such as ascorbic acid and/or insulin can be used to increase extracellular matrix production or to promote collagen growth.

Exemplary media or nutrients include but are not limited to one or more of the following: L-ascorbate acid or a phosphate derivative of L-ascorbate acid (e.g. Asc 2-P); serum; and growth factors, Dulbecco's Modified Eagle's Medium®(DMEM), DMEM F12 Medium®, Eagle's Minimum Essential Medium®, F-12K Medium®, Iscove's Modified Dulbecco's Medium®, RPMI-1640 Medium®, insulin, and/or ascorbic acid.

An exemplary cell suspension medium (e.g., before casting) is DMEM with Hepes.

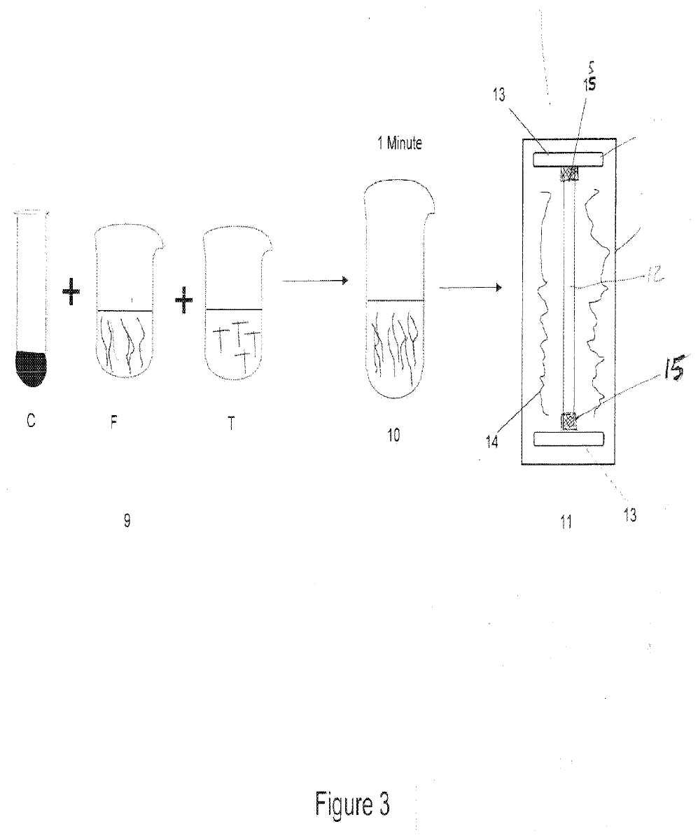

An exemplary cell suspension medium after casting, sometimes referred to as the 3D stage (also, FIGS. 3 and 4, steps 11-13), is DMEM with a growth factor supplement, preferably containing a serum albumin such as bovine serum albumin (BSA). Exemplary serum includes but is not limited to fetal bovine serum (FBS) or fetal calf serum (FCS) or formulated FBS substitutes such as Fetal Clone I, Fetal Clone II, Fetal Clone III, or the like, or a combination of any or all. In preferred embodiments of the invention, the cell suspension medium after casting also includes insulin and ascorbic acid.

After gel formation and an initial incubation period, the graft may be incubated in a growth or maintenance medium. This medium is typically supplemented with a medium such as FBS, and may optionally include one or more antibiotics, or growth factor. A preferred medium is one that does not include antibiotics. The most preferred growth/maintenance medium is DMEM with FBS (about 5% to about 40% concentration) and the antibiotic is a penicillin/streptomycin combination (sometimes referred to as “penstrep”). The medium may be changed and/or supplemented periodically as need. For example, this medium may be supplemented with formulated FBS substitutes, reduced FBS concentrations, insulin, and/or ascorbic acid.

In most cell culture applications, adherent cell cultures can only be maintained for a few days to a few weeks before the individual cells release from a substrate. The addition of agents that promote cell growth, viability and/or adhesion can be used during the culture process. For example, addition of agents such as ascorbic acid, retanoic acid, and copper can be used to increase the production of extracellular matrix proteins thereby generating a more robust tissue sheet of cells. Moreover, by treating the cell culture surface/substrate with extracellular matrix proteins or other factors (e.g., a protein such as gelatin or fibrin), adhesion can be prolonged.

The introduction of one or more tissue manipulation device(s)s (e.g., a plurality of control rods, polyester mesh, etc.) can be used to hold down the edges of the tissue on a culture container, form, or substrate, thus preventing spontaneous graft or tissue/material detachment. These devices or elements may also be used to control or limit tissue contraction. The rods or tissue manipulation device can be designed such that they generate clamping forces (e.g., via gravity, magnetic forces and the like) to effectively secure the tissue. Moreover, the rods can be made of a biocompatible material such as ePTFE (e.g., an ePTFE outer surface and a metallic core) or stainless steel that will be slightly adherent to the cells present in a tissue sheet. In this manner, once a tissue sheet is removed from the culture surface it can be handled easily.

In the most preferred embodiments, an exemplary illustration of which is shown in FIG. 4, a glass rod, having cuffs, preferably porous, on each end, is clamped into an incubator. During the initial tissue growth, tissue grows or embeds in the cuffs, which at this point are held or retained in place. This keeps the tissue taut, e.g., prevented from contracting. The rods are preferably non-adherent so the tissue can slide easily once the cuffs or ends are released. Preferably, the cuffs are porous so that the tissue can embed in the cuffs.

In some embodiments of the invention, the rods may be coated with pluronic acid to prevent adhesion to the rod. The rod also provides a surface upon which the tissue may be allowed to contract.

In some embodiments of the invention in which a non-tubular tissue is desired, e.g., a sheet or flat tissue, two or more rods, each having cuffs on each end, can be used to form the tissue between the rods like a scroll. In this way, the tissue could slide across or between the two rods, with the mesh ends keeping the tissue in place.

Decellularization

The cultured cells are then decellularized using any process that results in a decellularized RT. As used herein decellularized refers to a construct that is decellularized such that it is substantially acellular, immune-resistant, and/or calcification resistant. Biologically, there are various definitions of decellularization. At the time of this filing, the present inventors measure DNA and prefer that the starting DNA is less than about 3%. Others have described decellularization as the construct is substantially acellular if it comprises less than 2% cells, less than 1% cells or contains no cells. The cells are intact cells. The cells can be living cells or dead cells. Exemplary processes include but are not limited to: WO 2007/025233; WO 2010/120539; Ott, et al (2008, Nat. Med., 14:213-21); Baptista, et al. (2009, Conf. Proc. IEEE Eng. Med. Biol. Soc., 2009:6526-9); or Crapo, et al., (2011, Biomaterials, 32:3233-43). Other tests or measures may be used.

In another aspect, a tissue or material of the invention is substantially decellularized to provide extracellular matrix materials provided by the population of cells. In some cases, it may be advantageous to decellularize or denature all or part of the tissue or tissue engineered construct. A decellularized tissue may have a reduced level of immunogenicity, as well as other attributes. Decellularizing or denaturing the tissue may also enhance the mechanical characteristics of the tissue or construct. The tissue may be decellularized, denatured, or chemically modified using a variety of techniques. In the simplest embodiment, the tissue can be air-dried or lyophilized to kill the cells. Thermal shock, acoustic treatment, changes in pH, osmotic shock, mechanical disruption, or addition of toxins can also induce cell death or apoptosis. Similarly, the tissue can be cross-linked or fixed using agents such as paraformaldahyde. Other treatments to decellularize or denature the tissue are possible using radiation, detergents (SDS or triton ×100), enzymes (RNAase, DNAase), or solvents (alcohol, acetone, or chloroform). These techniques are only some of the examples of techniques to decellularize, denature or chemically modify all or part of the tissue and are not meant to limit the scope of the invention. Treatment with hypotonic and/or hypertonic solutions, which have non-physiological ionic strengths, can promote the decellularization process. Proteases also can be used effectively to decellularize tissue.

The decellularization can be performed in stages with some or all of the stages involving differential treatments. For example, a potent mixture of proteases, nucleases and phospholipases could be used in high concentrations to decellularize the tissue. The decellularized extracellular matrix may then have applied another tissue layer or another decellularized sheet.

The RT of the present invention may be characterized by one or more of the following: non-oriented fibers; oriented fibers; thickness up to about 1 mm, preferably between about 100 μm and about 500 μm; non-immunogenic or minimally immunogenic; a tissue, sheet or shape that is anisotropic; a tissue, sheet, or shaped structure produced by a process that includes scaled contraction (as described above); a sheet or shape that is suitable for cutting into shapes, e.g., by scalpel, die, or laser; suppleness; suturability; no or little calcification during life of implant; crosslink density, or variations of crosslink density through the material thickness; collagen concentration; collagen density, or variation of crosslink density through the material thickness; remodeling proclivity; absorption; resorption; degradability, regions of greater stiffness; and regions of greater flexibility.

In preferred embodiments of the invention, the RT has oriented fibers leading to suture pull-out resistance, anisotropic material properties as witnessed by tensile strength, even more preferably, adapted for its end use (e.g., a sheet, or a tube, or a valve).

### Various Method Steps:

Without intending to be limited to a particular process for producing RT, an exemplary method includes combining fibrinogen or the like, thrombin or the like, and extracellular matrix (ECM)-producing cells; and allowing the cells to grow sufficiently to produce ECM. One skilled in the art will recognize that up to nine cell passages typically optimizes ECM production. In preferred embodiments, the result is cells in a degradable suspension or gel.

The mandrel, shape, or form may be formed from a lubricious substance, or may be coated with a lubricious substance (e.g., teflon or pluronic acid); and the suspension/gel may be coated on the mandrel. In some embodiments, the gel suspension is a liquid or spray that fills into a mold. In preferred embodiments of the invention, the lubricious substance is a nonionic surfactant, such as pluronic acid.

In some embodiments of the invention, two opposing ends of the mandrel may include a slidable anchor (e.g., mesh) or the like that controls the time and amount of contraction of the gel. It has been found that as the tissue/suspension/gel contracts in a controlled fashion, fibers may orient in a certain direction. While on the form or substrate, the gel containing cells may be nourished by adding nutrient, growth, and/or maintenance medium, any or all of which may be chosen to promote ECM production.

Mechanical manipulations include, for example, static culture on a form (such as a isodiametric or non-isodiametric mandrel, glass sheet, or glass rod). Mechanical manipulation also includes moving the cuffs embedded with growing tissue along the rod (illustrated in FIG. 4, step 13). Allowing or purposefully shortening the axial length of the growing tissue leads to circumferential alignment as the cells compact the gel. In some embodiments, the tissue may be stretched or distended circumferentially in combination with axial shortening to maintain the circumferential alignment. Generally, such stretching or distortion may be cyclic or periodic. See, for example, Syedain et al., 2011, Biomaterials, 32:714-22. Simply by way of example, mechanical manipulations of the cell-seeded gel as it is remodeled into a cell-produced extracellular matrix can be performed using flow-stretch or pulsed flow-stretch methods in any number of bioreactors.

In accordance with the embodiments of the invention in which the fibers are aligned, the inventors have found that the amount of alignment may be scalable, e.g., the majority of fibers may be aligned in one direction (e.g., circumferentially), but there still are fibers aligned in different or other directions.

In an embodiment of the present disclosure, the implant, the fibers (of the matrix material) have a preferred orientation direction. Preferably, the fibers in the implant are arranged in such a way that when the implant is implanted, the fibers are arranged substantially perpendicular to the blood stream. Preferably, the preferred fiber alignment is circumferential around an imaginary axis of the implant wherein the axis points in the direction of blood flow in case of a tubular implant.

By mimicking the extra cellular matrix of the natural environment, a tissue can be grown having good structural properties, which eventually develop towards a native-like architecture (i.e., the tissues of the present invention are a biomimetic material).

Some embodiments of the invention may further include storing and/or sterilizing a medical device or tissue of the present invention. These embodiments may include preselected storage solution; preselected sterilization solution or technique; storage packaging; and/or sterilization packaging. In one embodiment, the tissue may be stored in PBS and refrigerated until use. Other storage/sterilization processes may include one or more additives known to those with skill in the art. In another embodiment, the tissue may be E-beam sterilized in PBS alone.

One skilled in the art will recognize that other storage and sterilization protocols may be used with the tissue of the present invention.

The RT may in turn be used to make a skirt, leaflets, a valve, or a coating or layer on medical devices intended for implantation. The RT of this embodiment may be used in any body lumen, including but not limited to an artery, a vein, or any body lumen that passes a body fluid (e.g., a ureter or a urethra). The present invention also is the use of the tissue as an implant. Suitable uses include but are not limited to coronary artery bypass graft (CABG), a valve leaflet, a peripheral graft, a sling, bulking agents, prolapsed bladder repair, stented or stentless pericardial valve replacement, stented or stentless pulmonic valve replacement, transcatheter valve prosthesis, aortic bioprosthesis/valve replacement or repair, annuplasty rings, bariatric surgery, dural patching, enucleation wraps, gastric banding, herniation repair, lung surgery e.g. lung volume reduction, venous valve replacement, pericardial patching, rotator cuff repair, uretheral slings, valve repair, vascular patching, valve conduit insertion, or arterial conduit insertion.

Preferred embodiments of the invention include a medical device or prosthesis of the present invention packaged and ready to use by the surgeon or in the operating room.

The inventors believe the following represent various mechanisms of action that may occur by following the processes of the present invention. Fibrinogen is a monomer that can be dissolved in saline and then mixed with cells to create homogenous suspension. The monomers are then crosslinked into polymer fibers using a catalyst. In some embodiments of the invention, thrombin is used as the catalyst, but other crosslinking agents may be used (e.g., ruthenium crosslinking chemistry).

At the outset of the fibrin gel formation, the composition or gel is primarily cells, fibrinogen, and residual proteins. As the process progresses, cells begin anchoring to fibers of fibrin and pull them leading to what is visually seems as gel compaction. The initial gel would be 99% water so mainly its water being squeezed out as cells pull fibers. Subsequently, cells produce extracellular matrix, while also producing chemicals that can clip fibrin fibers. As fibrin fibers are clipped, they are dissolved into surrounding media and washed out, leaving behind mostly cell produced extracellular matrix.

By restricting the gel from being pulled in all directions (e.g., controlled contraction), cells are forced to compact such that fibers end with orientation in a circumferential direction rather than being random. As cells produce extracellular matrix, the matrix is deposited in the same orientation as fibrin fibers via a mechanism called ‘contact guidance’.

Functionally, the inventors believe this organization of cell produced extracellular matrix rather than just random deposition by cells is the reason for superior performance of the resulting tissue.

Verification Protocols

One or more attributes of the RT or process conditions can be tested, determined, or evaluated using convention testing techniques. These verification protocols include, but are not limited to: visual observation and/or by product or process testing. Visual observation includes but is not limited to translucence, e.g., the tissue becoming more opaque and/or thicker. Product and process testing includes but is not limited to collagen content; tensile strength or modulus (in one or more directions, e.g., longitudinal or circumferential); suture retention (in one or more directions); histology; shrink temperature; acellular content; and thickness.

The present disclosure further relates to a method for growing a graft, comprising the step of providing an implant, preferably an implant according to the present disclosure to a subject (a patient). The implant is preferably an implant according to the present disclosure. This step can be preceded by the step of making an incision in the skin of the subject. In some embodiments of the invention, the graft is substantially isodiametric in a middle portion, shaped on one or both ends. In more preferred embodiments, the graft has at least one larger diameter end portion.

Some embodiments of the invention may include a degradable scaffold or stent that can be seeded by extracellular matrix producing cells. The scaffold may be formed from fibrin, PLA, PGA, or other synthetic or biological polymer, and mixtures thereof. The ECM producing cells can be cultured with the scaffold, allowing the cells to produce ECM, which can in turn replace the degradable scaffold. Optionally, the scaffold can be manipulated or processed (as described herein) to create alignment of the fibers in the ECM (e.g., an anisotropic matrix). The final product, preferably in the form of a sheet, may be decellularized using detergents, or dehydrated (e.g., freeze drying), to create a sheet of engineered tissue with or without cells.

The present invention also is a surgical kit comprising one or more of the following: a regenerative tissue implant or graft processed or produced according to the present invention; one or more instruments for implanting the graft; a rinse tray; a rinse solution, e.g., heparin; and suture material.

In preferred embodiments of the invention, the RT may be processed or formed using one or more tissue manipulation devices, some of which are described in U.S. Publication No. US 2007/0178588, filed Dec. 14, 2006, and incorporated by reference in its entirety.

The biological material may then be sterilized, following any sterilizing protocol e.g., by electron beam, gamma radiation, or the like. In a preferred embodiment of the invention, the biological material is sterilized by utilizing a liquid chemical sterilant consisting of 2% glutaraldehyde, or 75% ethanol and 2% propylene oxide at room temperature for approximately two weeks.

Finally, once the terminal sterilization step is completed (i.e. e-beam irradiation, gamma irradiation or the like), the prosthesis is ready for shipment and sale; alternatively, when using liquid sterilization techniques, the product is transferred from the sterilization media to its final packaging containing a sterile solution such as propylene oxide, for shipment and sale.

Alternatively, the tissue may be placed in its final or shipping packaging, along with appropriate solutions if needed, and sterilized, e.g., e-beam or critical CO₂ sterilization processes.

One of the embodiments of the present invention is a sterile closed package containing a biological material of the present invention. Typically, a separate container would hold individual or multiple samples having known size or dimensions. If desired, the biological material in the sterile package can be attached to another material or structure, such as an annuplasty ring, a sewing cuff, a synthetic graft, or a support for positioning the biological material on a stapler.

The biological material may then be placed or packaged in a container. In accordance with a preferred embodiment of the invention, the biological tissue is packaged and sealed in a bacteriostatic solution, typically in 1% propylene oxide, in its final container. Packaging preferably means placing the processed biological material in a container suitable for storage and/or shipping.

Methods of use include using the tissue configured to an appropriate shape as replacement tissue for any of the following surgical purposes: stented or stentless pericardial valve replacement, stented or stentless pulmonic valve replacement, transcatheter valve prosthesis, aortic bioprosthesis/valve replacement or repair, annuplasty rings, bariatric surgery, dural patching, enucleation wraps, gastric banding, herniation repair, lung surgery e.g. lung volume reduction, peripheral arterial or venous valve replacement, pericardial patching, rotator cuff repair, uretheral slings, valve repair, vascular patching, valve conduit insertion, or arterial conduit repair or insertion.

In peripheral application bypass embodiments, some of the embodiments may include one or more grafts above the knee, one or more grafts behind the knee, one or more grafts below the knee. A peripheral application embodiment may also be an AV graft, e.g., for use in dialysis.

In peripheral graft applications as well as other embodiments of the invention, the tissue may further include a latticework or stent positioned within the tissue, or the tissue may be grown around a latticework or stent.

In accordance with preferred embodiments of the invention, pre-determined shape refers to any forming or shaping the natural tissue into any shape or form that mediates or improves the hemodynamic function of the graft or its tissue reaction when it is implanted. These hemodynamic functions typically refer to blood flow dynamics within the graft after it is implanted. These dynamics include but are not limited to promoting laminar blood flow, reducing turbulence, minimizing shear at the anastomosis site, and/or protecting the bed of the recipient artery.

An embodiment of the invention includes a composite graft or tissue comprising regenerative tissue and one or more tapered synthetic portions (e.g., ends or cuffs).

In some embodiments, the invention is a prosthesis uses regenerative tissue formed into a graft, wherein said graft is formed into pre-determined shape. An exemplary shape is a tube or conduit, preferably hollow, or strawed shaped. In preferred embodiments, the tube has two ends and a central portion, and in the most preferred embodiments, the central portion is non-isodiametric in relation to one or both ends. For example, the central portion could be isodiametric and one or both ends of the tube could be a different shape, e.g., flared or cut. In some embodiments of the invention, at least one end of said prosthesis or tube is shaped into a cuff shape, including but not limited to the shapes shown in the Figures.

One skilled in the art will recognize that the processes steps described herein may be variously modified and a wide variety of ways in order to achieve a regenerated tissue with certain properties.

For ease and simplicity of describing concepts that form the basis of various embodiments of the invention, Applicants will describe several exemplary embodiments below in more detail. Applicants intend “exemplary” to mean that the description is only one or several embodiments of the invention. Other modifications to the invention may be made by one with skill in the art without deviating from the overall teaching of this invention. These other modifications are included within the teachings of this invention and application.

A very broad description of the various phases of the methods of the present invention may be described as including approximately five steps: cell culture; casting; media changes; decellularization; and sterilization and/or storage.

The cell culture phase refers generally to growing the initial cells. This phase also may include preparing various growth medias. Once the desired number of cells are grown, the cells are harvested and may be placed in a suspension used for casting.

The casting phase refers generally to creating a gel with cells suspended in it. This typically involves combining a pre-determined amount of cells (e.g., fibroblasts) with the fibrinogen solution and the thrombin suspension. One skilled in the art will recognize that the order of ingredients and speed of processing may be important—because fibrinogen and thrombin react so quickly together, the cells should be mixed with fibrinogen first. After the thrombin suspension is added, the cells become encapsulated in what quickly becomes a fibrin/thrombin gel. The cell/fibrin/thrombin gel is then transferred to a form or mandrel coated hydrophobic coating, which allows the gel to slide across the form without sticking to it.

The media changes phase refers to growing and maintaining the cells on the mandrel using conventional cell culturing techniques. As cells incubate, grow, and form a 3-D structure, the gel gradually becomes a tissue, eventually becoming a tissue that may be characterized as a cellular matrix.

The decellularization phase refers to washing and removing the cells from the cellular matrix. The result is a decellularized tissue. One skilled in the art will recognize that this phase follows conventional decellularization protocols.

The sterilization/storage phase involves processing the tissue in a wide variety of ways depending on its use and function.

An exemplary process of the present invention will now be described by reference to FIGS. 1-4. FIGS. 1 and 2 correspond generally to the cell culture phase. FIGS. 3 -4 illustrate the casting phase (steps 8-10), transitioning into media changes phase (steps 11-13), and into cellular tissue formation (steps 12-13).

FIG. 1 illustrates the initial cell growth and expansion steps. Frozen cells 1 are thawed and grown in appropriate media 2, plated and expanded in step 3, and grown through multiple passages in step 4. As shown, step 4 represents five passages (P5). After five passages, the cells may be optionally stored. All of these are well known culturing steps and processes.

FIG. 2 illustrates continued expansion steps until the final pass. Steps 6 and 7 show multiple expansion steps up to passage 7. As noted above, the inventors have found that about four to about 9 passages are sufficient to establish the number of healthy cells sufficient to produce regenerated tissue in accordance with the present invention. In step 8, the cells are combined and placed in nutrient media, now ready for casting stage. All of these are well known culturing steps and processes.

As shown in the casting stage (FIG. 3), in step 9 solutions of cells (C) produced in step 8, fibrinogen (F), and thrombin (T) are produced separately. Because fibrinogen and thrombin react together so quickly, the fibrinogen solution is first added to the cell solution; then the thrombin solution is added. Once the three primary ingredients are combined, step 10, gel formation begins almost immediately. Step 10 typically lasts about one minute, before the gel composition is transferred to an incubator (as illustrated, step 11).

FIG. 3, step 11 illustrates the transition between the casting stage and the media change phase. As shown, the incubator includes a glass rod 12 with caps 13 to seal/hold it in place. Rod or form 12 also includes moveable sleeves 15 at each end. Typical sleeves may be made of polyester mesh or the like, and are intended to resist tissue contraction along the rod (described in more detail below). The area on the rod/form between the sleeves has been previously coated with a hydrophobic coating (e.g., pluronic acid). The gel composition from step 10 is then combined with media and placed in contact 14 with the rod/form. Because the gel at this point is so delicate, the incubator is allowed to incubate without any movement of the incubator for about 30 minutes. Over time (after the initial 30 minutes), the cells produce extracellular matrix that solidifies or becomes more robust as the cellular matrix forms.

FIG. 4 illustrates the purpose of the sleeves 15 and additional processing steps that result in the final cellularized tissue matrix. The normal biological reactions that occur when cells, fibrinogen, and thrombin are combined and allowed to incubate result in a cellular matrix that contracts. This natural tendency, if allowed to continue, would allow the tissue to tear, form with holes or gaps, or form unevenly. The inventors have found that by controlling the natural contraction—specifically allowing the tissue on the form/rod to contract a certain amount—the developing tissue forms evenly and consistently.

For the purpose of this illustration, step 12 (at, for example, about day 6 of the incubation process) shows rod/form 12 with sleeves 15 positioned near the sealing caps 13. As shown, cell containing tissue matrix 16 grows around the form 12. Step 13 shows how the controlled contraction may be allowed to take place. In step 13, sleeves 15 are moved away from each cap along the coated rod/form 12.

In accordance with the present invention, one or more controlled contraction steps and one or more media changes may occur before the tissue is sufficiently ready to be decellularized. The choice of media, number of media changes, the number of contraction steps, the amount of contraction, the amount of time between contraction steps, the amount of time between media changes, and the amount of time for the overall process to run all depend on the specific cells used and the specific media used.

In accordance with some embodiments of the invention, the inventors have found that contraction in 10% FBS; with multiple contraction steps (typically one to about 3); with the time between contraction steps usually about one week apart; and with multiple media changes during each contraction phase (typically three) results in a cellular tissue of sufficient strength and character to be ready to be decellularized.

Further, the inventors have found that any materials that come into contact with cells, gels, graft material, or tissue is preferably minimally-reactive or non-reactive with the substance during the culturing step(s). The inventors have found that glass works effectively, but the invention should not be limited solely to the use of glass.

Also, the inventors have found that gentle handling of the growth, expansion, and mixing flasks; and the gels and incubators, etc. during the various process steps improves the success rate of the entire process. Gentle handling in moving the gel into the incubator is highly preferred.

### Definitions

The following definitions are used in reference to the invention:

(A) As used herein, “decellularization” refers to the process of removing cells from a blood vessel, such that the three-dimensional structure of the extracellular matrix (ECM) scaffold remains. Physical methods and chemical and biologic agents are used in combination to lyse cells, often followed by a rinsing step to remove cell remnants and debris. Effective decellularization is dictated by factors such as tissue density and organization, geometric and biologic properties desired for the end product, and the targeted clinical application. Decellularization of blood vessels with preservation of the ECM integrity and bioactivity can be optimized by those skilled in the art, for example, by choosing specific agents and techniques during processing.

A variety of decellularization processes may be used. An exemplary process is described in U.S. Publication No. 2007/0178588, incorporated herein by reference.

As indicated herein, a decellularized vessel consists essentially of the extracellular matrix (ECM) components of the vascular tree. ECM components can include any or all of the following: fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina. Successful decellularization is defined as the absence of detectable myofilaments, endothelial cells, smooth muscle cells, and nuclei in histologic sections using standard histological staining procedures. Preferably, but not necessarily, residual cell debris also has been removed from the decellularized organ or tissue.

(B) As used herein, biomimetics or biomimicry refer to imitating the models, systems, and elements of nature for the purpose of solving complex human or animal problems. In the present invention, biomimetics is used for therapeutic purposes.

### EXAMPLES

### Example 1

### Casting Protocol

### Reagents:

20 mM Hepes; CaCl2, fibrinogen; thrombin; 5% pluronic acid; DMEM w/Hepes; DMEM w/FBS and w/Penstrep; D/F12 Media, insulin; and ascorbic acid.

### Method:

Coat form (e.g., mandrel; mold; tube; glass plate or rod) with a lubricous substance, e.g., pluronic acid solution.

If tissue contraction or stabilization is needed, prepare the devices or elements needed to stop or control contraction. These devices or elements may include restrictors or elements that resist or prevent the tissue from contracting, or allow controlled or limited contraction.

Harvest cells

Mix fibrinogen solution (fibrinogen stock into 20 mM Hepes).

Mix thrombin solution (thrombin stock into DMEM w/Hepes and CaCl₂ solution;

Resuspend cells in DMEM w/Hepes without FBS or serum supplement.

Add cell suspension to fibrinogen solution.

Add thrombin solution to cell suspension/fibrinogen solution, inject suspension into mold within 1 minute; do not move for about 5 minutes, then transfer to incubator and incubate to allow gel to solidify (e.g., for about 30 minutes). As used herein, a graft is formed or begins to form as soon as the clotting reaction begins, typically within about one to two minutes after the cell/fibrinogen solution is mixed with the thrombin solution.

Supplement culture media DMEM base with FBS, P/S (optional), insulin, and ascorbic acid, as needed. (second medium).

Carefully release the graft from the mold and place in a culture dish with second medium.

### Example 2

### Growth and Shaping Protocol

The graft is then fed and/or shaped as the collagen content increases and the fiber alignment and cross linking takes place. The graft culture media is changed periodically as needed using the second medium (Penstrep optional).

The graft is matured until the desired endpoint. In this experiment, the endpoint is determined by both visual observation and/or by product or process testing. Visual observation includes but is not limited to translucence, e.g., the tissue becoming more opaque and/or thicker. Product and process testing includes but is not limited to collagen content; tensile strength or modulus (in one or more directions, e.g., longitudinal or circumferential); suture retention (in one or more directions); histology; shrink temperature; acellular content;, collagen content, residual DNA and thickness.

During this maturation phase, various other steps may be incorporated into the protocol: mixing, detachment, and contraction.

If the culture medium needs to be mixed, the medium may be agitated, rocked, or shaken.

If collagen fiber alignment is desired, the graft may be contracted one or more times by allowing the graft to contract along the form, or by gently manually moving the graft along the form. In this example, manual manipulation was used—rings holding the tissue from contracting are moved to create slack. The cells then contract and tighten the tissue.

Once the maturation and shaping phase has been completed, the graft (still supported on its form) is then ready to be decellularized.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

While the invention has been described in some detail by way of illustration and example, it should be understood that the invention is susceptible to various modifications and alternative forms, and is not restricted to the specific embodiments set forth in the Examples. It should be understood that these specific embodiments are not intended to limit the invention but, on the contrary, the intention is to cover all modifications, equivalents, and alternatives falling within the spirit and scope of the invention.

1. A process for producing regenerative tissue comprising combining extracellular matrix-producing cells with fibrinogen monomer and a polymerization catalyst, and allowing a fibrin gel to form; allowing the gel to be compacted by cells; allowing the fibrin to degrade to produce a cell-containing and ECM-containing tissue; and decellularizing the tissue, thereby forming a regenerative decellularized tissue.

2. The process of claim 1 wherein the extracellular matrix-producing cells are fibroblasts.

3. The process of claim 1 wherein the polymerization catalyst is thrombin.

4. The process of claim 1 wherein the cells produce ECM and produce chemicals that clip fibrin fibers.

5. The process of claim 1 wherein allowing a gel to form further comprises placing the gel in contact with at least one shape or form.

6. The process of claim 5 wherein the form is a rod.

7. The process of claim 5 wherein at least one end of the form is shaped.

8. The process of claim 1 wherein allowing the gel to form a tissue further comprises controlling the contraction of the tissue.

9. The process of claim 8 wherein controlling the contraction of the tissue further comprises controlling fiber alignment in the tissue.

10. The process of claim 1 further comprising sterilizing the regenerative tissue.

11. The process of claim 1 wherein the regenerative tissue is suitable for implantation in a human or animal.

12. The process of claim 11 wherein the regenerative tissue is a graft, a bypass graft, a sheet, a patch, a conduit, a bulking agent, a sling, an AV graft, or one or more portions of any of the above.

13. The process of claim 1 wherein the regenerative tissue is a prosthesis suitable for implantation in a human or animal.

14. The process of claim 1 wherein the regenerative tissue is used to make a prosthesis suitable for implantation in a human or animal.

15. The process of claim 14 wherein the prosthesis is a valve, a heart valve, a venous valve, a mono-cuspid valve, a bi-cuspid valve, a tri-cuspid valve, or one or more portions of any of the above.

16. The process of claim 5 wherein the form is a stent and the tissue forms a coating around the individual elements forming the lattice of the stent.

17. A tissue made by the process of claim 1.

18. An implant made using the regenerative tissue formed by the process of claim 1.
